(19) United States
(12) Patent Application Publication
(10) Pub. No.: US 2004/0259177 A1
(43) Pub. Date: Dec. 23, 2004

(54) Three dimensional cell cultures in a microscale fluid handling system

(76) Inventors: Robert G. Lowery, Madison, WI (US); John Majer, Madison, WI (US); David J. Beebe, Madison, WI (US)

Correspondence Address: Sara D. Vinarov, Quarles & Brady LLP, P.O. Box 2113, Madison, WI, 53701-2113, US

(21) Appl. No.: 10/839,916

(22) Filed: May 6, 2004

Related U.S. Application Data
(60) Provisional application No. 60/468,358, filed on May 6, 2003.

(51) Int. Cl. G01N 33/574; C12M 1/34

(52) U.S. Cl. 435/7.23; 435/287.2

(57) ABSTRACT

The present invention provides a novel microscale fluid handling system for initiating, culturing, manipulating and assaying three-dimensional multicellular assembly. The system including a microfluidic device and three-dimensional multicellular tissue surrogate assembly. The device of the invention includes at least one microfluidic channel; and at least one chamber, wherein the walls of the chamber are lined with a cell layer; and wherein fluid medium flows through each of the channels and chambers. Also, disclosed are methods for using the device to introducing test agents to the multicellular assemblies to observe biological responses thereof.

### CROSS-REFERENCE TO RELATED APPLICATIONS

This application claims the benefit of U.S. Provisional Patent Application Ser. No. 60/468,358, filed May 6, 2003, which is incorporated by reference herein in its entirety.

### STATEMENT REGARDING FEDERALLY SPONSORED RESEARCH OR DEVELOPMENT

Not applicable.

### TECHNICAL FIELD

The present invention relates to spheroids in a microscale fluid handling system. The present invention provides a device and methods for initiating, culturing, and manipulating three dimensional (3D) multicellular surrogate tissue assemblies, such as spheroids, culture media, extra cellular matrix components, soluble signaling molecules and cell-to-cell interactions. The present invention further provides high throughput screening (HTS) methods to study agents capable of intervening in diseases modeled by spheroids in a microscale fluid handling system.

### BACKGROUND OF INVENTION

Mammalian cell culture has been traditionally used as a model for studying disease processes, especially cancer, and testing of potential therapeutic agents used in treatment thereof. Generally, cells used for mammalian cell cultures are grown in monolayers on plastic plates covered with liquid medium, which supplies essential nutrients and growth factors for the cells. However, for most cell types this method of culturing does not adequately mimic the in vivo environment from which the cells were originally isolated (1). This is because disease pathogenesis occurs in the context of 3D tissue structures, and involves interactions between different cell types in the stromal and epithelial compartments and with the extracellular matrix (ECM) (1). Not surprisingly, cells grown in monolayers often do not exhibit the same biological responses and behaviors that they would otherwise in an in vivo environment. In contrast, cells grown in spheroid structures and in the presence of extra cellular components that simulates their normal environment generally represent an in vivo biological environment much more faithfully (1). Accordingly, three dimensional multicellular aggregate, such as: spheroids, mammospheres, organoids, and organotypic cultures offer great potential for improved in vitro disease models that may be used to screen and develop novel therapeutic agents.

A spheroid is a 3D aggregate of living mammalian cells cultured in vitro from tissue explants, established cell cultures or a mixture of both. Spheroid research, initially, focused largely on monoculture of cells as 3D aggregates. However, recently heterologous spheroids with more than one cell type have been used to investigate the interactions of different cell types in both normal tissue and tumor development (1). The internal environment of a spheroid is dictated by the metabolism and adaptive responses of cells with a well-defined morphological and physiological geometry. Beyond a critical size (>500 uM) most monotypic spheroids develop concentric layers of heterogeneous cell populations with proliferating cells at the periphery and a layer of quiescent cells close to the necrotic core (1). This heterogeneous arrangement of cells in a spheroid mimics initial avascular stages of early tumors. Another type of monotypic spheroid forms well organized acini-like structures with a central lumen when epithelial cells are cultured over reconstituted basement membrane (2). These monotypic spheroids are able to mimic important in vivo morphology, although much of the biological complexity is lost. With the co-culture of more than one cell type in a spheroid, tumor cell interactions with other cell types can be studied under standardized conditions. Both the normal tissue and tumor micro milieu can be better defined as the epithelium, its underlying basement membrane and the sub-adjacent stroma. Co-culture of tumor cells and normal endothelial cells, as spheroids, has been useful for studying angiogenesis. The co-culture of tumor cells with stromal elements including stromal fibroblasts has demonstrated the importance of the complex micro-environment in neoplastic progression. Normal stroma was shown to inhibit tumor cells, while stroma from tumor biopsies was shown to have a mitogenic effect on tumor cells (2).

Spheroids have already made contributions to the general understanding of tumor biology and normal tissue development. In fact, the importance of soluble signaling molecules, cell-to-cell signaling and the influence of ECM on tumor progression has been elucidated using spheroid tumor models (1, 3). Compared to homogeneous monolayer cell culture methodologies, these in vitro tumor models preserve many biochemical and morphological characteristics, which correspond to in vivo tumors (3). Furthermore, in vitro tumor models provide a useful method of testing the influence of etiological and potential therapeutic agents. For example, it has been demonstrated that in vitro breast tumor models respond to estrogen stimulation, a key factor in the etiology of the disease in vivo (3).

Furthermore, a vast body of oncology research has shown the importance of mutations that activate dominant oncogenes and inactivate tumor suppressor genes (4). However, these studies have focused on the cancer cell alone, while overlooking the complexity and heterogeneity of the whole tumor. This cell autonomous perspective describes cancer as a progressive set of genetic alterations that drive the transformation of normal cells to highly malignant tumor cells. However, it is equally important to understand neoplastic progression as increasingly abnormal signaling between the different cell types and between the cells and the ECM in the tumor microenvironment (2). Accordingly, spheroids may provide a more realistic in vitro tumor model than traditional monolayer cultures where these signaling abnormalities may be analyzed and the effects of anti-cancer agents may be readily evaluated. The importance of modeling tumorigenesis using 3D cell culture methods is well illustrated in the case of breast cancer.

Mammary glands are composed of a network of epithelial ducts supported by a dense stroma, which accounts for more than 80% of breast volume. The ducts are formed by an inner layer of polarized epithelial cells, an outer discontinuous layer of myoepithelial cells and a sheathing of specialized ECM called basement membrane as illustrated in FIG. 1. The stroma contains fibroblasts, endothelial cells, inflammatory and other specialized cells imbedded in a macromolecular network of ECM. The basement membrane and stromal ECM are composed of different combinations of collagen, laminin and other glycoproteins and proteoglycans that mediate binding and signaling to epithelial cells via transmembrane integrin proteins. The ECM provides both architectural support to cells and contextual information that influences their response to external stimuli for growth, differentiation, and motility.

Breast tumors originate in the epithelial cells of terminal duct lobular units, and it is well established that the accumulation of mutations and chromosomal aberrations within these cells are central to tumorigenesis. It is the tissue microenvironment (FIG. 2), however, that defines and controls, the cell-ECM interactions that define mammary tissue architecture—polarized epithelial cells bounded within the confines of a basement membrane—are subverted, allowing the tumor cells to invade the stromal compartment, grow, and metastasize. Much of the cellular signaling controlling this process occurs via cell surface receptors known as integrins, which bind to components of the ECM, and whose expression and distribution is frequently altered in malignant cells (5). Integrins modulate intracellular signaling pathways that control cell proliferation and apoptosis and they regulate the activity of extracellular proteases involved in invasion and metastasis. Signaling between different cells—known as paracrine signaling also plays a key role in mammary tumorigenesis. Aberrant paracrine signaling by steroid hormones and polypeptide growth factors—both intraepithelial and stromal-epithelial are responsible for many aspects of malignancy in the breast (6, 7), and the effects of these hormones are integrated with cell-ECM interactions (8). Furthermore, recent data on stromal mutations in mammary tumors suggests that the genetic underpinnings of carcinogenesis may be stromal, as well as epithelial in nature (9, 10).

To overcome the lack of phenotypic differentiation observed in monolayer cultures, 3D cell culture methods incorporating a basement membrane have been developed (11). For example, when grown in the presence of a reconstituted basement membrane, normal and malignant human mammary epithelial cells form 3D structures with clear morphological and biochemical differences that reflect their in vivo phenotypes (12). Normal cells form organoids similar in their overall organization to mammary acini: polarized epithelial cells surrounding a central lumen. Normal cells also deposit surrounding basal lamina (even in the presence of the reconstituted basement membrane) and cease growing after they reach a diameter of 40-50 μm (12). In contrast, malignant cells form solid, disordered masses similar to tumors that continue to grow to much larger sizes and do not secrete a basement membrane. The differences between the growth and differentiation patterns of normal and malignant cells are distinguishable only when cells are grown in the presence of a matrix rich in collagen and laminin or Matrigel™ that provide the ECM components necessary to direct tissue architecture. Such 3D-reconstituted basement membrane (3D-rBM) culture methods are an improvement over monolayer cell culture because they incorporate cell-ECM interactions important to tumorigeneis. However, still these methods do not account for stromal cells, and thus, do not reflect stromal-epithelial signaling that occurs in native mammary tissue.

Recently, mammary epithelial cells and various types of stromal cells have been incorporated into heterotypic spheroids (13, 14) and used to study aspects of tumorigenesis involving paracrine signaling. In one model, tumor cell and fibroblast spheroids were grown separately, then combined and allowed to merge; the tumor cells eventually enveloped and invaded the fibroblast spheroid (15). In another experimental model, tumor cells (epithelial) were cocultured with fibroblasts and/or endothelial cells in the presence of reconstituted basement membrane (16), an extension of the 3D-rBM culture methods described above. In this system, a mutual interdependence between tumor cells and endothelial cells for estrogen dependent ductal morphogenesis and neovascularization was observed (16). In similar experiments, fibroblasts isolated from tumor fibroblasts were shown to be necessary and sufficient to induce morphogenesis of both normal and malignant epithelial cells, and these effects were further enhanced by the addition of endothelial cells (17).

Another approach that has been used to recapitulate the dynamics of tissues, specifically mammary tissue has been to coculture epithelial and stromal cells in adjacent ECM layers (18, 19). A layer of fibroblasts in collagen is overlaid with epithelial cells in collagen or reconstituted basement membrane. This provides a two compartment system for studying interactions between the lower “stromal” layer and the upper epithelial layer. This model has been used to study how estrogen dependent proliferation of epithelial organoids is mediated via growth factors produced by fibroblasts (18), and to study the role of fibroblast-produced growth factors and proteases in epithelial organoid branching (19).

Although, both of the models described above establish the feasibility of recapitulating paracrine signaling between stromal and epithelial cells in vitro, both systems also have shortcomings. Heterotypic spheroids are basically disordered masses of cells and thus, bear little resemblance to the ordered structure of mammary tissue. In addition, there is no clear separation between epithelial and stromal compartments with these models. The two compartment coculture methods are an improvement on this approach in that they incorporate separate epithelial and stromal compartments. However, the “bulk” nature of classical tissue culture methods used for both models limits the ability to control experimental variables and to monitor the activities of the system at the scale of the tissue microenvironment. Once the intact cocultures are established, any changes made to the system are global. Test agents must be added directly to the liquid medium that overlays the coculture, exposing the entire system to the agents with no control of how their effects are changed by different cell types in either compartment. Also, it is not possible to specifically stimulate the epithelial or stromal compartment with a test agent. In addition, exposure of the system to a constant concentration of test agent for a defined period of time is not practical with these models either, as it would require frequent aspiration and replacement of the liquid overlay which is both cumbersome and stressful to cells. These physical constraints limit the ability to experimentally probe the system and to mimic the paracrine signaling and compartmental control systems that operate in vivo.

More generally, current methods for the initiation and analysis of spheroids involve labor-intensive processes and are not easily amenable to the high degree of standardization and automation that are required for routine drug screening. Furthermore, current methods such as static cell culture and flow through cell culture generally subject the spheroids to mechanical stresses and it is difficult to control the microenvironment around the cell mass. Static culture methods fail to allow for the gradually changing milieu in the normal tissue or tumor microenvironment. Flow through culture methods use large fluid volumes and the medium is replenished so quickly that important growth factors and other biological signaling molecules are washed away.

Furthermore, it has been established that at the micro-scale different forces become dominant over those experienced at larger scale (20) these include laminar flow, diffusion, fluidic resistance, surface area to volume ratio, and surface tension. Laminar flow is the definitive characteristic of microfluidics. Fluids flowing in channels with dimensions up to several hundred microns in width and at readily achievable flow speeds are characterized by low Reynolds number, (Re). Flows in this regime are laminar, not turbulent. The surfaces of constant flow speed are smooth over the typical dimension of the system, and random fluctuations of the flow in time are absent. In the long, narrow geometries of microchannels, flows are also predominantly uniaxial. The entire fluid moves parallel to the local orientation of the walls. A suitable feature of uniaxial laminar flow is that all transport of momentum, mass, and heat in the direction normal to the flow is left to molecular mechanisms: molecular viscosity, molecular diffusivity, and thermal conductivity.

Microfluidics allows for precise and unique control of the local fluid environment as well as the ability to work with smaller reagent volumes and shorter reaction times. Microscale phenomena enable techniques and experiments not possible on the macroscale. For instance, the laminar flow properties of microchannels are such that the mixing between two streams flowing in contact is diffusion dependent—i.e., not affected by turbulence mixing factors. This makes it possible to generate concentration gradients and discrete packets of reagents for use as stimuli to biological systems.

The first microfluidic devices were fabricated in silicon and glass by conventional, planar fabrication techniques—photolithography and etching—adapted from the microelectronics industry. These methods are precise, but expensive, inflexible, and poorly suited to exploratory work. Recently new techniques such as soft lithography, in situ construction, micro-molding and laser ablation have been applied to the fabrication of microfluidic devices (20). These nonphotolithographic microfabrication methods are based on printing and molding organic materials, and are much more straightforward than photolithography for making both prototype devices and special-purpose devices for physical investigations. These methods have also made it practical to build 3D networks of channels and components (21). Thus, they may offer access to new types of fluidic elements, such as valves and pumps fabricated of elastomeric materials (22). In addition, they offer the high level of control over the molecular structure of the channel surfaces that is required in many applications. To date use of spheroid cell cultures in a drug discovery setting has been limited because existing methodologies used for their growth and manipulation have not allowed accurate reconstruction of tissue morphology, specifically in the signaling between stromal and epithelial cells. Accordingly, it would be desirable to provide alternative approaches using microfluidics to precisely manipulate the micro-environment of a 3D cell culture.

### SUMMARY OF THE INVENTION

The present invention is summarized as a microscale fluid handling system comprised of a microfluidic device and a three dimensional (3D) multicellular assembly of living cells, wherein the device is used for initiating, culturing, manipulating, and assaying the multicellular assembly, preferably at least one spheroid.

One aspect of the present invention provides a microfluidic device for initiating, culturing, manipulating, and assaying multicellular surrogate tissue assemblies including at least one microfluidic channel; at least one chamber; and at least one spheroid, wherein the walls of the chamber are lined with a cell layer; and wherein fluid medium flows through each of the channels and chambers.

In another aspect the invention provides a microfluidic device for initiating, culturing, manipulating, and assaying multicellular surrogate tissue assemblies having two adjacent chambers are lined with a cell layer, wherein each chamber contains a spheroid representing a different tissue, and wherein each chamber contains a fluid medium specific for a tissue.

In another aspect the invention provides a method of performing high throughput screening of test agents using surrogate tissue assemblies by making a microfluidic device including fluid flow channels and chambers; making surrogate tissue assemblies of multiple cell types of mammalian cells; placing surrogate tissue assemblies, preferably spheroids into chambers in the device; introducing test agents through the fluid flow channels to the surrogate tissue assemblies; and observing the responses of the surrogate tissue assemblies.

In another aspect the invention provides a high throughput screening system for mimicking the reaction of multicellular tissues to test agents. The system includes a microfluidic device having a plurality of fluid flow channels and a plurality of chambers; and a plurality of surrogate tissue assemblies formed of living mammalian cells, each surrogate tissue assembly located in one of the chambers.

Still another aspect of the invention encompasses providing kits having a microfluidic device of the invention and spheroids used to study agents capable of intervening a variety of medical conditions.

These and other aspects of the present invention would be better appreciated upon an examination of the following drawings, description, taken in conjunction with the appended claims.

### BRIEF DESCRIPTION OF THE DRAWINGS

FIG. 1 shows the structure of human mammary tissue. Mammary ducts are composed of polarized epithelial cells surrounded by a discontinuous layer of myoepithelial cells encased in a specialized cell layer (ECM) layer called the basement membrane. The ducts are imbedded in a dense layer of stroma composed of ECM proteoglycans, glycoproteins and specialized cell types including fibroblasts, macrophages, adiopocytes, and endothelial cells.

FIGS. 2A-B show flow patterns inside the microfluidic channels. Two streams flowing in contact will not mix except by diffusion. As the time of contact between two streams increases, the amount of diffusion between the two streams increases. The top and bottom portions of spheroid 1 are exposed to different test agents with a sharp boundary across the equator and spheroid 2 is exposed to a gradient from top to bottom (A). Fluid flow in direction 1 with minimal leakage into the perpendicular channel. Fluid is then allow to flow in direction 2 to move a packet of fluid out of stream 1 and down the channel exposing the spheroid to a short pulse of soluble factors in the fluid packet (B).

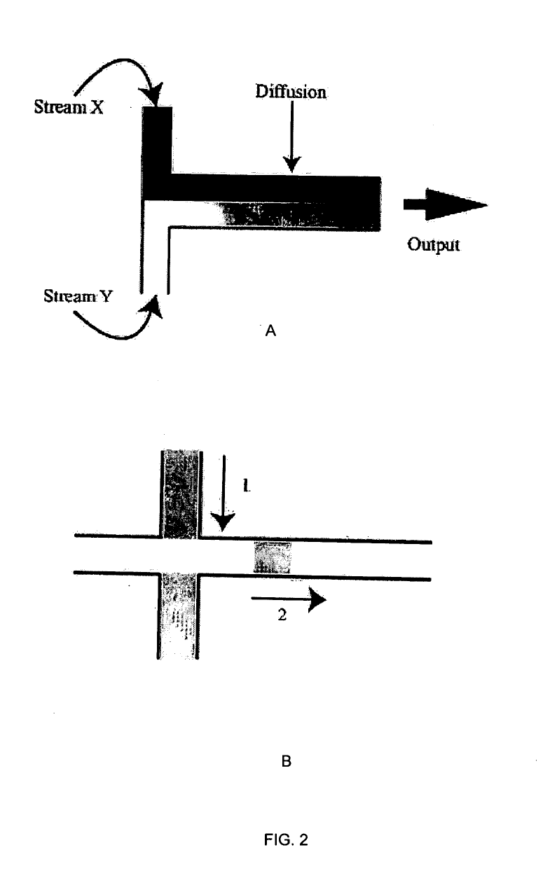

FIG. 3 shows the top view of a narrow section of the channel that allows the fluid, but not the spheroid to pass.

FIG. 4 shows the cross-sectional view of an obstacle in the bottom of the channel that prevents the spheroid from traveling any further.

FIG. 5 shows spheroids attached to a fibroblast seeded microchamber.

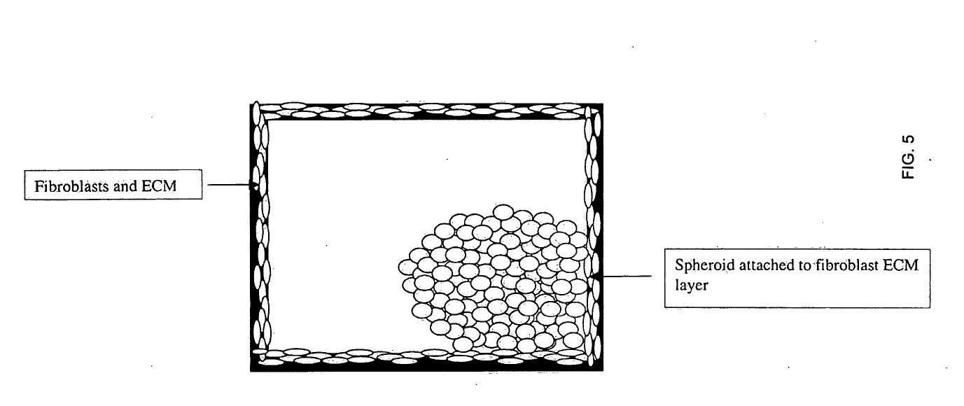

FIGS. 6A-B show spheroid behavior in culture flasks and microchannels. In culture flasks spheroids lie at the bottom of a layer of media. Any established micro-environment will diffuse away because of the large volume of media (A). In microchannels, there is much less media surrounding spheroids, thus reducing the effects of diffusion on the micro-environment (B).

FIGS. 7A-C show spheroid development and organization. Cross sectional view of spheroid where concentric growth pattern mimics early avascular tumor (A). Spheroid comprised of mammary epithelial cells grown over reconstituted basement membrane to induce mammary acini like structure development (B). Heterotypic spheroid showing formation of ducts and vascular elements with epithelial cells and basement membrane surrounding the stromal core (C).

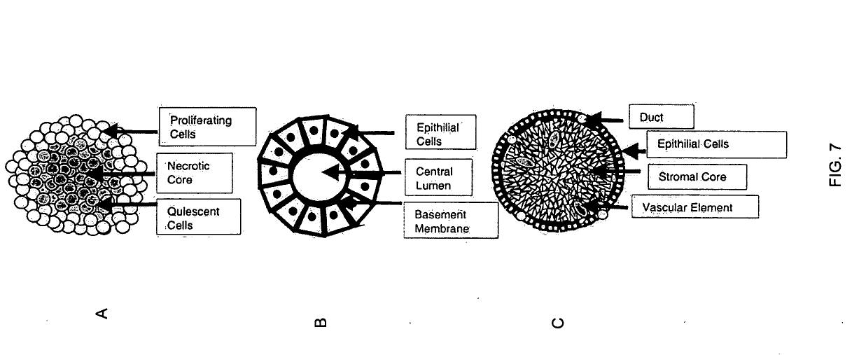

FIG. 8 shows a portion of a microscale device with multiple channels and chambers providing identical microenvironments for many spheroids and individual testing and sampling capabilities.

FIG. 9 shows latitudinal cross sectional views of chambers showing different ways of distributing equal numbers of cells or spheroids of a uniform size.

FIG. 10 shows longitudinal cross-sectional view of channels and chambers showing how basement membrane and spheroids can be introduced to chambers.

FIGS. 11A-B show a schematic of a microfluidic device for coculture of epithelial organoids and stromal fibroblasts in adjacent compartments (A) and T-junction that is used to allow delivery of discrete pulses of reagents (B). Arrows indicate the direction of fluid flow through the channels of the device. The two compartments are formed sequentially by flowing cell/collagen suspensions into the incubation chamber via the upper channel and increasing the temperature to allow collagen to gel. The stromal layer is supported by a microporous filter (dashed line). The upper and lower channels provide the ability to separately control the exposure of each compartment to factors of interest.

FIGS. 12A-B show a detailed drawing of a microfluidic device for coculture of epithelial organoids and stromal fibroblasts in adjacent compartments, specifically (A) side view and (B) top view. The three layers of material are indicated by brackets; the numbers indicate the sequence of fabrication. Each layer is approximately 300 μm thick. The epithelial and stromal fluid channels and the incubation chamber with a filter in the bottom is fabricated using liquid phase photopolymerization of PEG diacrylate. The filter supports both layers of cells embedded in ECM (collagen) and allows the free passage of soluble molecules. The layers of cells suspended in ECM are introduced via the epithelial fluid channel and allowed to gel in the device. Arrows indicate direction of fluid flow in channels.

Before an embodiment of the invention is explained in detail, it is to be understood that the invention is not limited in its application to the details set forth in the following description. The invention is capable of other embodiments and of being practiced or being carried out in a variety of ways. Also, it is to be understood that the phrases and terminology used herein is for the purpose of description should not be regarded as limiting in any way.

### DETAILED DESCRIPTION OF THE INVENTION

We have developed a novel microscale fluid handling system which combines a microfluidic device to a three dimensional (3D) multicellular assembly of living cells. The device is used for modeling a medical condition by initiating, culturing, manipulating, and assaying the 3D multicellular assembly of living cells, preferably a spheroid. The invention also provides methods for using such a device to model disease progression by assaying test agents for stimulation, inhibition or prevention of neoplastic progression in a spheroid. The method includes high throughput screening of the agents capable of intervening in diseases modeled by spheroids in a microscale fluid handling system; detecting an acquired spheroid characteristic; and assaying acquired spheroid characteristic to select agents for stimulation, inhibition or prevention of neoplastic progression, suitably mammary cancer.

As used herein, the term “three dimensional” or “3D” cell culture refers to any method used to effect the growth of cells into a 3D multicellular r surrogate tissue assembly or spheroid; includes organotypic cell culture methods that are used to effect the growth of cells into their native tissue morphology.

Also, as used herein, the term “spheroid” refers to an aggregate or assembly of cells cultured to allow 3D growth as opposed to growth as a monolayer. It is noted that the term “spheroid” does not imply that the aggregate is a geometric sphere. The aggregate may be highly organized with a well defined morphology or it may be an unorganized mass; it may include a single cell type or more than one cell type. The cells may be primary isolates, or a permanent cell line, or a combination of the two. Included in this definition are mammospheres, organoids, and organotypic cultures; and more specifically, the well ordered acini-like organoids formed by mammary epithelial cells in certain culture conditions.

In general in vivo tissue development and homeostasis rely on carefully orchestrated “cues” from soluble signaling molecules, attachment factors in the ECM, and cell-to-cell signals. The term “ECM” or “extracellular matrix” refers to a cell layer composed of different combinations of collagen, laminin and other glycoproteins and proteoglycans that mediate cell binding and signaling. The ECM provides both architectural support to cells and contextual information that influences their response to external stimuli for growth, differentiation, and motility. Includes native ECM, plain collagen, synthetic mixtures, and natural isolates (i.e., Matrigel™).

Both the temporal and spatial coordination of these cues is critical for creating an in vitro model of the normal tissue. Compared to other mammalian cell culture systems, one embodiment of the present invention provides a microscale fluid handling system, which provides an environment which more faithfully models in vivo conditions for growth and differentiation by allowing precise control of soluble signaling factors in the fluid medium, cell attachment surfaces, pressure, pH, and cell-to-cell communication.

In this embodiment, the invention also provides a means for manipulating the micro-environment of a 3D multicellular surrogate tissue assembly. As used herein, the term “manipulating” or “manipulate” refers to the ability to precisely control the micro-environment of a 3D cell culture, including the pH, hydrostatic pressure, gradients, flow rate, introduction of soluble factors representing endocrine and paracrine signals, cell-to-cell interactions, and specific ECM components.

Specifically, the invention provides for gradual changing of the fluid medium in a precise manner representative of an in vivo environment. Current methods for culturing suspended spheroids (23) require transferring the spheroid in a pipette. Pipette transfer can shock the spheroid with sudden changes in local environment and expose the spheroid to mechanical stresses that are not representative of the in vivo environment. Another method of changing media for both suspended and attached spheroids is flow through culture. Standard flow through culture dilutes and washes away exogenous signaling molecules that are important to normal tissue differentiation and development.

Also, it is envisioned that the laminar flow properties of the channels used in the present invention enable unique assays, not possible using macro-scale tissue culture methods. Two or more laminar flow streams can be joined into a single multi component laminar flow stream. This allows researchers to expose different portions of a single spheroid cell culture to different soluble factors simultaneously or to establish gradients across the spheroid cell culture. Test compounds, signaling molecules, or enzymes can be delivered in discrete packets within the laminar flow stream allowing precisely timed exposure of the spheroid cell culture. The channel geometry and flow rate define the temporal and spatial aspects of the exposure of the packet to the spheroid. The physiology and state of differentiation of cells at different depths within the spheroid plays an important role in tissue differentiation (24). The ability to peel layers off the spheroid cell culture allowing assays for morphological characteristics and surface markers of cells at different depths within the cell mass may provide insights into their state of differentiation (23). Further the introduction of discrete packets of enzymes associated with both normal processes of tissue differentiation and tissue invasion or metastasis may allow assays to further elucidate the role of these enzymes (2) in tumor progression. It is also possible to combine chemical treatments (e.g. fluid packets) with mechanical manipulation. For example, suction through small ports has been used to vacuum the cumulus cells off of bovine oocytes. Similar manipulations could be used to selectively remove layers of cells from spheroids.

Furthermore, it is envisioned that obstacles within the channels can hold a suspended spheroid in place while maintaining a continuous or pulsed flow of medium across the spheroid (25). The spheroid can easily be moved out of these holding places by reversing the fluid stream (26). A series of obstacles within the channels can also serve to sort the spheroids by size. The first obstacle in the series prevents spheroids larger than the opening around the obstacle from entering the culture channel. At the downstream end of the channel a second obstacle holds the desired spheroids in place while allowing smaller spheroids to exit the culture channel.

In another embodiment of this invention, the microfluidic device is capable of supporting spheroid cultures with basement membrane and ECM components. The ECM consists of macromolecules secreted by cells into their immediate microenvironment. These macromolecules interact to form an insoluble matrix. The ECM can serve as the scaffolding on which cells migrate, or it may induce differentiation in certain cell types. Some macromolecules forming the ECM include for example, collagens, proteoglycans, and substrate adhesion molecules. The basement membrane is a thin layer of insoluble macromolecules interposed between cells and the adjacent connective tissue. In the capillaries, basement membrane forms a boundary between the endothelial lining of the blood vessel and the adjacent mesenchyme. Macromolecules forming the basement membrane include for example, collagen IV, laminin, and proteoglycans. The basement membrane has a supportive function in some tissue and may also act as a passive selective filter (27). It is envisioned that microfluidic channels used for the initiation of spheroid cell culture may be coated with biopolymers that are important components of the ECM and basement membrane. These biopolymers may include, but are not limited to laminin, fibronectin, gelatin and collagens. Matrigel™ (Collaborative Biomedical Products, Catalog No. 40234) a synthetic basement membrane preparation may also be used to prepare the culture channels.

In addition to the ECM, it has become clear that cell to cell signaling is a vital part of both normal and neoplastic differentiation and development (8). Diffusible factors like growth factors, hormones, and morphogens are secreted by one cell type to change the behavior of other cell types. Cells can also selectively recognize other cells based on cell surface properties causing some cells to adhere and others to migrate past each other based on affinity. These affinities can be for the surfaces of other cells or for components of the ECM. The dominant paradigm of morphogenesis is differential cell affinities to localize cells appropriately within tissues, organs and tumors (27).

Furthermore, the progression from normal tissue to malignancy can be characterized by increasingly abnormal communication between cells that comprise the tumor and the tumor microenvironment. In this context, tumor formation can be considered a developmental process where a complex organ forms in response to signaling between different cell types and the ECM. It has been demonstrated that targeted expression of stromelysin-1, a matrix metallo-protease, produced spontaneous acquisition of tissue features characteristic of neoplastic states (2). Increasingly abnormal communication between fibroblasts, endothelial, and epithelial cells has been shown to induce processes such as tumor angiogenesis using in vitro tumor models (3). As used herein, the term “tumor model” refers to an in vitro cell culture system used to mimic the behavior of a malignant tumor.

Additionally, the present invention provides methods for seeding microfluidic channels with different cells types to initiate spheroid cultures. Specifically, it is envisioned that the present invention provides methods to seed biopolymer coated channels with fibroblasts, which are the predominant cell type found in the stromal compartment. It is further envisioned that the growing fibroblasts will condition the channel by adding their own ECM components to the biopolymer coating. The fibroblast-conditioned channels will then be seeded with spheroids containing epithelial cells, and possibly additional cell types.

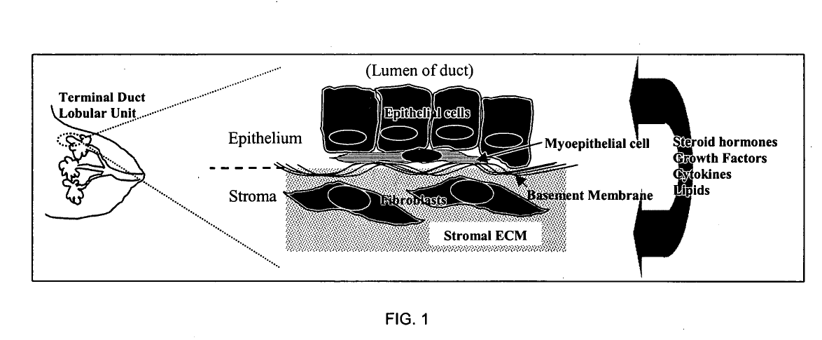

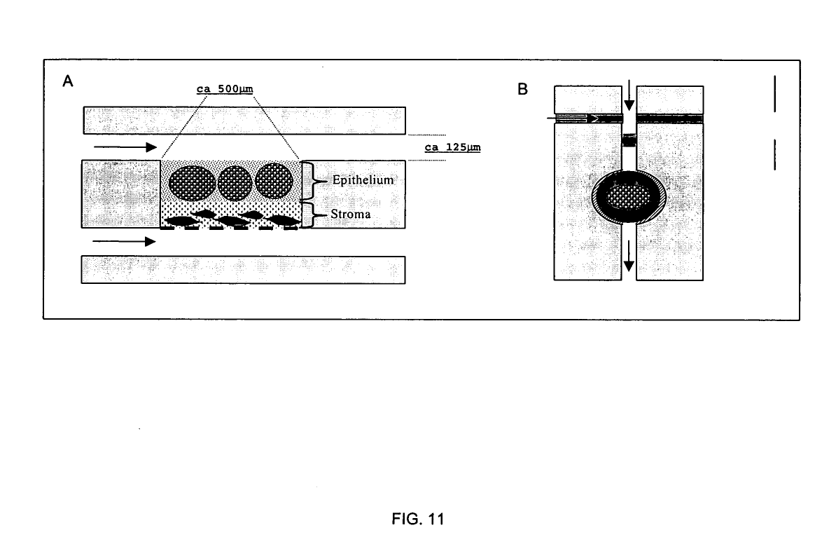

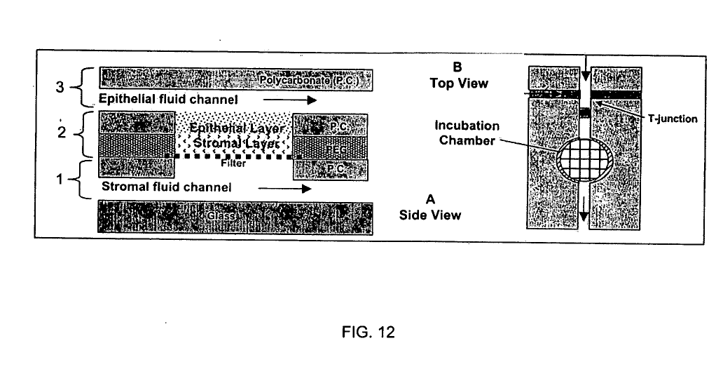

The present invention also provides a microfluidic device designed to incorporate stromal and epithelial cells in adjacent compartments as shown in FIG. 11A and FIG. 12A, mimicking the structure of a mammary tissue in vivo (FIG. 1). An example of such a device is shown in FIG. 11, which illustrates how two tissue compartments may be separately addressed by two different solvent streams. Specifically, FIGS. 11A-B show a schematic of a microfluidic device for coculture of epithelial organoids and stromal fibroblasts in adjacent compartments (A) and T-junction that is used to allow delivery of discrete pulses of reagents (B). Arrows indicate the direction of fluid flow through the channels of the device. The two compartments are formed sequentially by flowing cell/collagen suspensions into the incubation chamber via the upper channel and increasing the temperature to allow collagen to gel. The stromal layer is supported by a microporous filter (dashed line). The upper and lower channels provide the ability to separately control the exposure of each compartment to factors of interest.

It is believed that the ability to selectively probe either tissue compartment is a significant improvement over other two compartment models described earlier, because it will enable simulation of stromal-epithelial signaling more accurately. For instance, it will be possible to selectively initiate signals in one compartment and monitor the response of the other.

It is envisioned that polymers sensitive to stimuli including pH, light, temperature, biological signals, and electrical current (22) may be incorporated into the microfluidic channels. Subtle changes in external stimuli can cause the hydrophilic polymer to expand or contract exerting or relieving pressure on spheroids growing in the microfluidic device. Furthermore pressure may be applied to an elastomeric membrane adjacent to spheroid cell cultures. The rate and extent of deformation can be measured and controlled using particle imaging (28). Development of organs and tumors in vivo is often responsive to pressure exerted by the surrounding tissues. Therefore, in accordance with the invention, we envision that pressure sensitive polymers and elastomeric membranes may be used in the device of the invention to control pressure on the in vitro tumor.

In each case, parallel control channels within the microfluidic device may be used to compare spheroids exposed to test agents with control spheroids exposed to the same growth conditions but without the test agents. The control channel has a similar structure and is part of the same fluid control system used for the test channels so that flow rates and cell culture media are identical during the course of the experiment.

In another embodiment, the present invention provides methods for measuring growth, proliferation, differentiation, and development of spheroids. Observation of spheroid size, cell shape, and developmental features such as angiogenisis, and duct formation can often give information on its state of differentiation. Morphological analysis may be carried out using an inverted microscope to analyze spheroids in place or by standard histological techniques, as well as image analysis of specific cell markers (23). Fluorescence labeling of cells, organelles, or macromolecules using exogenous fluors or expressed fluorescent proteins, such as green fluorescent protein, may be useful for detecting changes in spheroid properties. Proliferation may be measured directly using a number of methods including but not limited to the MTS colorimetric method (Promega Corporation, Madison, Wis.).

An attached spheroid culture may be dissociated from the channel using a trypsin or pronase solution and the suspended spheroid may be extracted for further analysis outside of the microfluidic device. The fluid media may also be assayed for soluble factors. The microfluidic channels do not dilute soluble factors to the extent seen in standard culture techniques and the ability to precisely control a pulse of fluid across the cell culture allows factors such as enzymes, hormones, and growth factors to be washed out of culture in a more concentrated form (20). Some of these soluble factors would include growth factors and proteases. Enzyme linked immunosorbent assays (ELISA) may be used to determine the presence or quantity of growth factors. Metallo-proteases are often an indicator of tissue differentiation or tissue invasion and Zymogram gels (Invitrogen, Carlsbad, Calif.) are useful in measuring this activity.

In the search for new therapies pharmaceutical companies screen vast libraries of compounds for their ability to increase or inhibit specific enzyme activities or binding to specific nuclear receptors. Many of these assays involve measuring a change in absorbance, fluorescence or nuclear magnetic resonance (NMR) properties of reporter molecules in a high throughput screening mode in parallel arrays, where the characteristics of spheroids between channels are constant so that many agents may be tested for effects on essentially identical spheroids. Accordingly, in another embodiment, the invention provides a means to establish spheroid cell cultures in channels that are compatible with 24, 48, or 96 well format currently used for drug candidate screening. It is envisioned that biochemical assay reporter molecules can be introduced into the microfluidic culture channels or produced by cells in the spheroid and direct measurements of change in the reporter molecule could be taken directly from the microfluidic device (29). This may provide a rapid method for verifying that compounds showing desired biochemical properties during initial screening and a corresponding inhibition or promotion of spheroid development are actually functioning as predicted in the spheroid.

### EXAMPLES

Producing a plurality of multicellular surrogate tissue assemblies (e.g., spheroids) of a uniform size and overall structural properties using a microscale device is an important capability because it allows one to test diverse chemicals for their effects on essentially identical spheroids. The prophetic examples below describe methods used to produce multiple spheroids of a similar size and overall set of characteristics, isolated from each other in separate addressable chambers, and thus can be subject to different experimental variables. There are two approaches that can be used: initiation and growth of spheroids in the microscale (MS) device, or use of the MS device to sort and distribute spheroids from bulk cultures grown outside the device.

### Example 1

### Initiation and Growth of Similar Sized Spheroids in a MS Device

In this prophetic example, cells are grown initially in plates as monolayers, then enzymatically detached, collected and introduced to a microscale (MS) device with multiple channels leading to chambers where the spheroids will form. This is done in such a way that equal numbers of cells are distributed to each chamber, insuring that the size of the spheroids that form will be uniform if maintained under identical culture conditions. Distributing cells to multiple chambers can be achieved in several ways, including introducing a uniform cell suspension through a single opening that branches to multiple channels each leading to a chamber, using concurrent flow through each channel or using a valve that opens onto each channel separately.

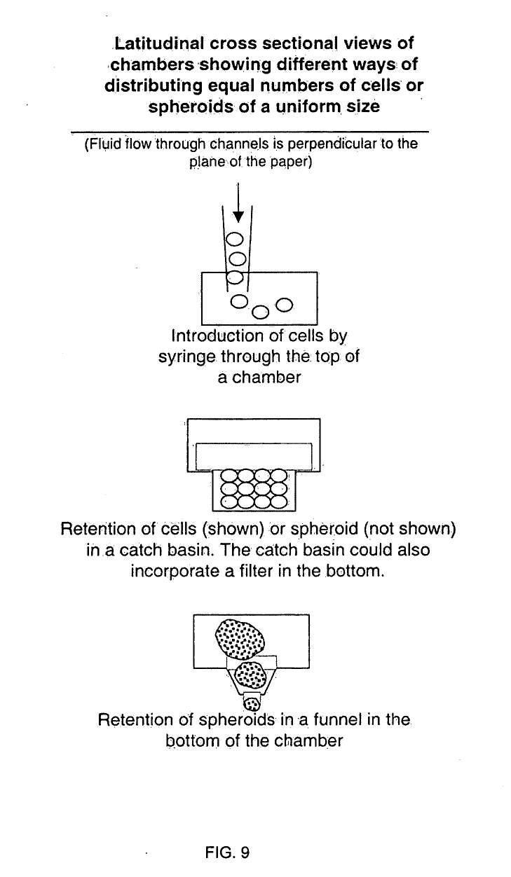

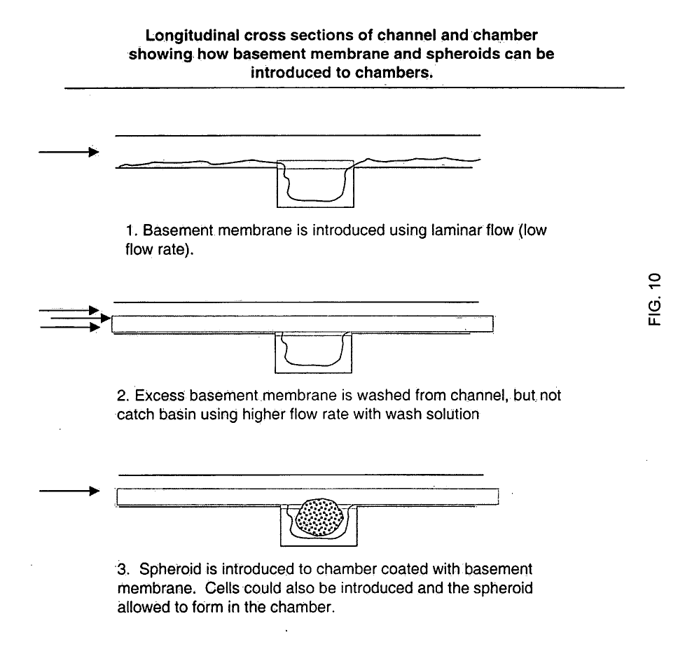

Alternatively, an equal volume of a uniform cell suspension is introduced through separate openings for each channel, or through separate openings directly into the chambers. The cells can be introduced via a manual syringe, or a pneumatically operated syringe with single or multiple outlets, or by an automated liquid handling device such as is commonly used for dispensing biological reagents in an HTS setting. Spheroid culture chambers will be coated with a stationary non-adherent layer of agarose (23) or other biopolymer. Distributing an equal or nearly equal, number of cells to each chamber can be achieved in several ways, including maintaining constant flow rates for the same period of time for introduction of cells to each channel and then using valves to shut the chamber off from fluid flow while excess cells are washed out of the channels. Alternatively distribution of equal number of cells to chambers is achieved by filling a depression or catch basin in the chamber. In this latter case, cells are introduced at a low flow rate such that laminar flow properties occur until the chamber is filled, then the channels are cleared of excess cells by flushing out the channels with media or a wash solution at an increased flow rate, such that the fluid path is uniformly horizontal and does not enter the catch basin as illustrated in FIGS. 9 and 10. In a variation of this approach, the catch basin can include a filter in the bottom that retains cells such that laminar flow is out the bottom of the catch basin.

### Example 2

### Distributing Spheroids of a Uniform Size to Chambers for Further Growth and Analysis

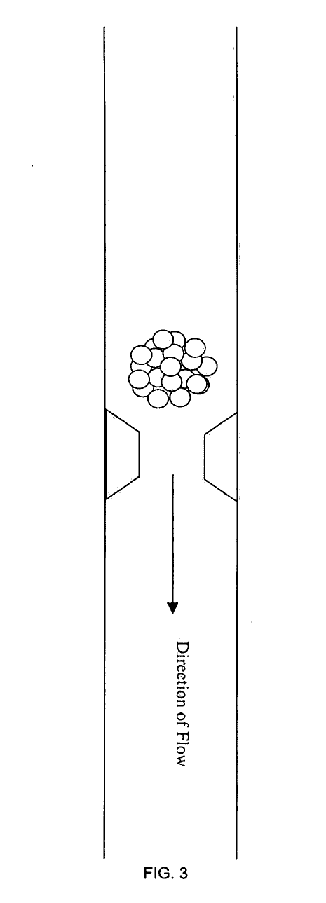

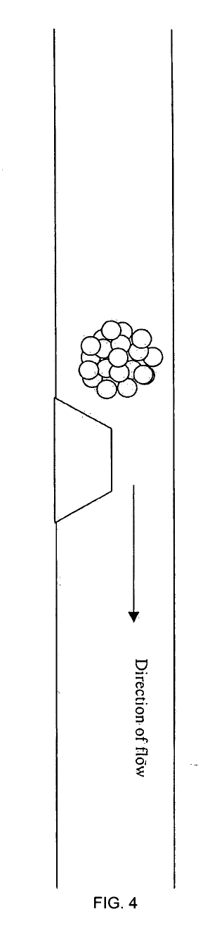

As an alternative to initiating spheroids in the MS device, it is also envisioned that spheroids may be grown by standard cell culture methods—on agar plates or in spinner flasks—and distribute them to the chambers of the microscale device using fluid flow. Spheroids of a uniform size can be attained by the use of physical structures such as filters, funnels or barriers in the fluid path that act as sieves. For example, FIGS. 3 and 4 illustrate the presence of such physical structures or obstacles in a channel of the microfluidic device, enabling the fluid, but not the spheroid to pass. Also, FIG. 9 shows latitudinal cross sectional views of chambers showing different ways of distributing equal numbers of cells or spheroids of a uniform size. Thus, if a funnel is used, all spheroids larger than the desired size pass through the chamber because they are unable fit in the large opening of the funnel, and those that are smaller than desired pass through the small end of the funnel.

### Example 3

### Culturing Spheroids

Also, prophetically exemplified here are a variety of techniques for culturing spheroids. Once spheroids or surrogate tissue assemblies are in chambers, they are cultured by flowing media through the chamber, such that nutrients are replenished, and waste products are removed. The flow rate is slow enough to allow growth factors and other soluble signaling molecules in the spheroid microenvironment to carry out their functions before they are washed away from the spheroid. In this way, an essentially identical microenvironment is maintained in each chamber, allowing spheroids to form and grow at the same rate and develop similarly. In some cases, it may be desirable to change the type of media or some components at some point in the development of the spheroid. If desired, some chambers can be subject to different growth conditions or soluble factors in order to test their effects of spheroid formation. It may also be desirable also to culture spheroids in the presence of basement membrane components and/or stromal cells such as fibroblasts or immune cells. If this is the case, basement membrane and stromal cells are flowed into the chambers prior to introduction of the intact spheroid or spheroid cells.

### Example 4

### Analysis of Spheroids

In accordance with this invention, it is envisioned that the cultured spheroids described above may be analyzed either for size, or gross morphological properties is generally done using microscopy, and for this purpose, spheroids can be observed while still in the MS device.

Alternatively, if a soft polymer matrix such as PDMS is used for casting MF device, the spheroids in their individual chambers can be excised from the device using a boring or sectioning device and subject to detailed morphological and histological analyses either in whole, or following thin sectioning, fixing, enzymatic digestion, staining, and/or other tissue and cell preparation methods. Alternatively, the spheroids can be released individually from their chambers using enzymatic digestion of basement membrane and ECM matrix and their contents collected for analysis. Inhibition or stimulation of the spheroid cell proliferation may be measured directly using the MTS colorimetric method (Promega Corporation, Madison, Wis.). The MTS reagents may be introduced through the common fluid handling system and the change in absorbance measured directly from in the MS device. In addition, many other experimental outputs can be measured, either in the MS device, or following release of the spheroid. These include outputs that give an indication of the status of tumorigenesis, such as gene expression patterns, presence of specific enzyme activities or cell surface receptors, secretion of soluble factors, etc. Methods for measuring these outputs are well established and include immunochemical, DNA or RNA hybridization, the use of reporter proteins, and the use of reporter substrates, and involve mostly colorimetric, luminescent and fluorescence detection methods.

### Example 5

### Spheroids as a Model for Cancer

There is a developing body of literature describing the use of spheroids as in vitro tumor models (2, 3). Both monotypic and heterotypic spheroids have proven useful as tumor models. Heterotypic spheroids offer the ability to investigate interactions between different cell types in the tumor microenvironment (3). Monotypic spheroids comprised of malignant cells offer the advantage of simplicity and they can effectively represent initial avascular stages of early tumors. Monotypic spheroids may be prepared by seeding single cell suspensions of tumor cells on a stationary nonadherent layer of agarose. After 3 to 8 days the spheroids reach a size of 300 to 400 nm.

It is prophetically contemplated that in experiments to test new drug therapies it is important that the spheroids are all of the same size because small differences in spheroid diameter have a dramatic effect on the volume and morphological characteristics. Thus, the spheroids of the invention will be sorted by size using a series of obstacles before the spheroids analyzed.

### Example 6

### High Throughput Screening of Test Agents

Also, prophetically exemplified here are methods for high throughput screening (HTS) of etiological agents that stimulate tumors and potential drugs to suppress tumors. It is envisioned that a microscale fluid handling device with will be used to analyze spheroid cultures, and this device will be compatible with existing instrumentation for measuring absorbance, fluorescence, luminescence or other signals used to quantify biological responses in an HTS setting. The analysis device may be the same as the device used to initiate and/or grow the spheroids, or it may be a separate device that spheroids are transferred prior to analysis. Spheroids for testing and analysis will be present in chambers in the MS device in a pattern that is consistent with existing multiwell plates, including but not limited to 24, 96, or 384-well plates.

Alternatively, new instrumentation might be developed that is more suitable for analysis of spheroids in microfluidic devices. The chambers will be depressions or wells in the channels or alternatively the chambers will be sequestered using barriers or walls. The depressions will be coated with reconstituted basement membrane, and possibly also seeded with fibroblasts and/or other stromal cell types creating an extra cellular matrix to mimic in vivo conditions. Monotypic spheroids comprised of a tumor cell line will be sorted for size and spheroids within a specific size range will be deposited in chambers within the channels. The spheroids will attach to the basement membrane and their growth and morphology will be monitored by microscope. When the spheroids reach an optimum size for testing, test agents will be introduced to the non-control spheroids. All of the chambers share a common fluid handling system and each chamber can be addressed separately. One way to achieve this is by the use of separate ports and channels leading to each chamber, another is by the use of valves. The non-control spheroids will be exposed to test agents while both control and non-control spheroids are exposed to the same flow rates and biological media. Inhibition or stimulation of the spheroid cell proliferation may be measured directly using the MTS colorimetric method (Promega Corporation, Madison, Wis.). The MTS reagents may be introduced through the common fluid handling system and the change in absorbance measured directly from the analysis stations using the 24, 48 or 96 well format currently used for drug candidate screening.

In addition, many other experimental outputs can be measured that give an indication of the status of tumorigenesis, including but not limited to gene expression patterns, presence of specific enzyme activities or cell surface receptors, and secretion of soluble factors. Alternatively, if a soft polymer matrix such as PDMS is used for casting MF device, the spheroids in their individual chambers can be excised from the device using a boring or sectioning device and subject to detailed morphological and histological analyses either in whole, or following thin sectioning, fixing, enzymatic digestion, staining, and/or other tissue and cell preparation methods. Alternatively, the spheroids can be released individually from their chambers using enzymatic digestion of basement membrane and ECM matrix and their contents collected for analysis.

### Example 7

### Fabrication of a Two Compartment Device for Reconstructing Mammary Tissue Using Liquid Phase Photopolymerization

With the recent development of liquid phase photopolymerization, the entire design and fabrication cycle for a microfluidic device has been reduced to minutes, making it possible to produce and test many devices during the prototype development process (30). Such an iterative approach is particularly well suited for building a device that will house complex biological systems as described by the present invention.

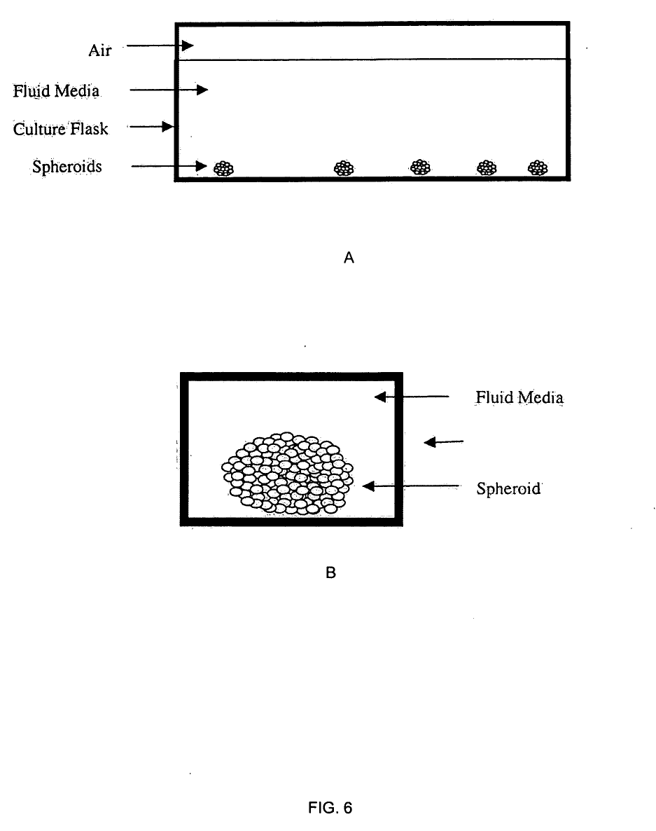

It is envisioned that to construct a microfluidics device, a prepolymer solution is flowed into a chamber and exposed to UV light through a mask that prevents photopolymerization where channels or other openings are desired. Uncured prepolymer is subsequently flushed from the channel. The sides and bottom of the chamber are formed by an adhesive gasket adhered to a microscope slide and the top is a polycarbonate film (FIG. 6). The adhesive gasket maintains the cavity height, (increments of 125 μm), and the flexibility of the polycarbonate top accommodates the inherent shrinkage of the polymer solution. Small holes are prepunched in the polycarbonate layers for access to fluid channels. Multilayered devices are constructed by repeating this fabrication process—with whatever channel configuration is desired—on top of the preceding layer. Interconnections between the horizontal channel layers are achieved by through holes in the photopolymer that align with prepunched holes in the polycarbonate tops; these holes become the sites for input and output ports on the top layer. In this manner, any number of layers can be fabricated, one on top of the next.

It is envisioned that porous polycarbonate filters will be integrated in the device shown in FIG. 12. The filters in the device is used to physically support the two ECM/cell layers while allowing free diffusion of soluble molecules. The first layer is punched with a through hole in the center and a channel network is formed underneath. Before making the second layer, the filter is placed on top of the through hole and secured with glue to the surface. The upper channel is then built around the filter using the same layering technique as described earlier.

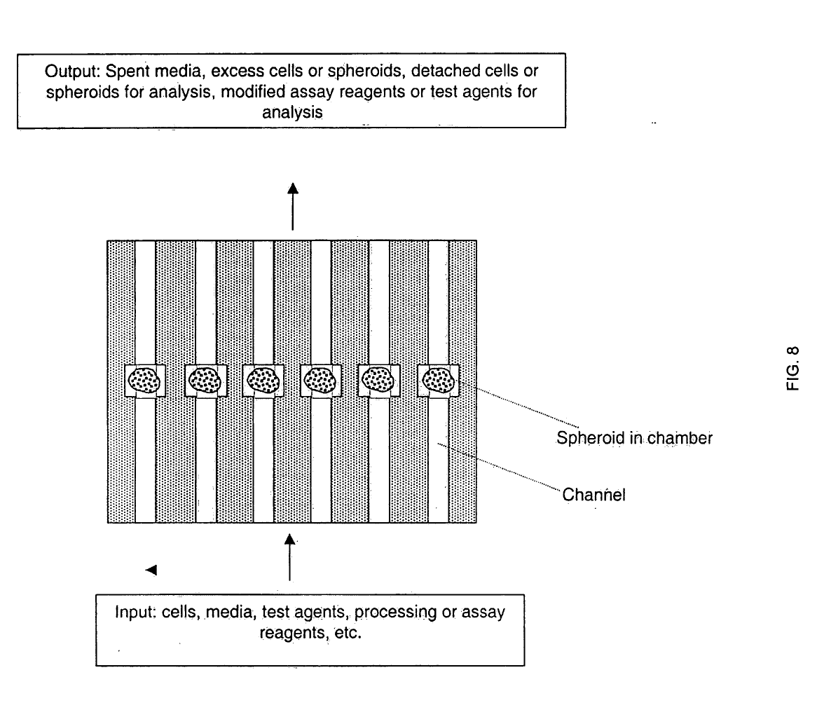

Furthermore, it is envisioned that liquid phase photopolymerization will be used to fabricate the three-layered design shown in FIG. 12, and these are used assembly of stromal-epithelial cocultures. Polyethylene glycol diacrylate is used as a prepolymer and 4-(2-hydroxyethoxy) phenyl-(2-hydroxy-2-propyl) ketone (Irgacure 2959, Ciba, Inc.) as a photoinitiator for the photopolymerization process; both of these components are have been validated for biocompatibility with mammalian cells (31, 32). Briefly, the three layers of the device, comprised of polymerized PEG and a flexible polycarbonate top, are fabricated in sequence starting with the bottom layer on a glass slide (FIG. 8). For each layer, the liquid prepolymer is introduced by syringe into the chamber formed by the polycarbonate top resting on a perimeter gasket (Hybriwell, Grace BioLabs, Bend, Oreg.). A mask with the desired pattern for the channel blacked out is placed over the chamber, the exposed prepolymer is irradiated with UV light (360 nm, 2-10 s at 20 mW/cm²), and excess prepolymer is flushed from the channels with distilled water. A 5 μm pore size polycarbonate filter (Osmonics) is incorporated into the bottom of the incubation chamber in the second layer with glue. All three layers of the device are fabricated before introduction of ECM or cell/ECM mixtures. The collagen matrices used are identical to those used for actual cell culture, including Vitrogen-100 (Cohesion Corp, Palo Alto, Calif.) and Matrigel™ (Collaborative Research, Inc., Waltham, Mass.).

### Example 8

### Coculture of Mammary Epithelial Cell Organoids and Stromal Cells in Separately Addressable Compartments of the Microfluidic Device

Another prophetic example, envisioned by the applicants is the ability to coculture mammary epithelial cell organoids and stromal cells in separately addressable compartments of the microfluidic device. It is believed that MCF10A and its sublines can be used as the mammary epithelial cell line because it has been used extensively for 3D-culture (11), and is relatively simple to maintain. MCF10A is a spontaneously immortalized cell line isolated from a woman with fibrocystic breast disease, and is one of only three human mammary epithelial cell lines considered to be non-malignant (33). A number of MCF10A sublines that have been made malignant by the introduction of oncogenes such as H-ras and Erb-B family members (21, 33, 34, 35). Use of these sublines allows comparison of organoid behavior using genetically matched normal and malignant cells. There are no commercially available human mammary fibroblasts cell lines of stromal origin, and most of the basic research in this area is done using primary isolates. If obtaining and processing human tissue and maintaining primary cultures is too difficult, NIH-3T3 cells—a very well characterized mouse fibroblast cell line—can be used. Also, a human fibroblast line that was derived from normal breast skin (CCD-1086Sk, ATCC No. CRL2103) is a reasonable replacement for primary fibroblast cultures. The CCD-1086Sk cells are not immortalized, but are capable of at least 23 doublings. NIH3T3 cells are maintained as monolayer cultures on 100 mm plates in DMEM with 10% bovine calf serum, and passaged once per week. MCF10A cells are maintained as monolayers in DMEM/F12 media supplemented with fetal bovine serum, growth factors, and antibiotics (33, 36), and passaged twice per week.

The formation of separate stromal and epithelial layers containing mixtures of ECM and cells with proper size into the incubation channels is achieved by using a T junction (FIG. 11B). Briefly, a cooled aqueous collagen/cell mixture (collagen remains liquid at 4° C.) is flowed into the latitudinal channel and pressure is applied to the longitudinal channel to push and separate a collagen/cell packet with the size determined by the design of the T junction. The packet is then directed (by creating a flow between the upper inlet and lower outlet ports) into the incubation chamber that is formed at the connection between the upper and lower channels where the packet is allowed to gel at 37° C. Prior to gelling, the collagen is prevented from flowing through the filter by prefilling the lower chamber with liquid to the bottom of the filter. In typical 3D culture constructs, 1-2 ml of collagen solution will gel in about 15 mins at 37° C. However, in microchannels the volumes used are smaller by several orders of magnitude (0.1˜0.5 μl), thus faster gelation due to improved heat transfer can occur. Other control parameters are the concentration, pH, temperature, and dimensions of microchannels. If necessary, to provide more precise control of the gelation process, additional heat exchange channels can be included in the design to provide precise spatial and temporal control of the thermal conditions.

Furthermore, the desired final format for the two compartment device described above is a layer of ECM containing stromal cells such as fibroblasts adjacent to a layer of ECM containing epithelial organoids, or acini, which mimic the morphology of mammary terminal lobular ducts (see FIGS. 1 and 11).

To recapitulate the in vivo structure of the mammary gland, two gel compartments are required. Thus, the process described above is performed first to form the stromal compartment, and then repeated to form a second epithelial compartment on top of stromal compartment. Because the gelation of collagen is controlled by the pH, temperature, and concentration, it is feasible to introduce another package of epithelia-collagen without disturbing the first compartment. The second packet (epithelial cells mixed with collagen) is introduced via the T-junction as described above and brought into contact with the stromal compartment formed previously and allowed to gel. Because fluid flow through the first collagen layer is restricted, one relies primarily on density sedimentation for deposition of the top layer of collagen. Different collagen concentrations are tested to find the optimal combination of viscosity and density. After the two compartments are formed they each can be fed from separate channels: the epithelia culture media and reagents can be introduced via the upper channel and fluids for the stromal compartment can be introduced via the bottom channel. Thus, the overall structure of the system allows for the independent exposure of each compartment via the two channels.

For establishing the stromal compartment, different approaches—including allowing fibroblasts to adhere first and overlaying with ECM or adding cells in an ECM suspension can be optimized for a particular stromal cell line. Filters of different materials and pore sizes can also be tested for cell attachment. For establishing 3D organoids of MCF-10A cells in the device, well described methods for 3D culture of mammary epithelial cells (33, 11, 36) in conventional tissue culture apparatus are adapted. The basic protocol involves dissociating monolayer cell cultures and resuspending them in a commercially available ECM material called Matrigel™ (Collaborative Research, Inc., Waltham, Mass.), which is liquid at low temperatures, but gels at 37° C. Confluent monolayer cultures of MCF10A are dissociated with trypsin-EDTA for several minutes at 37° C., pelleted by centrifugation, and resuspended in DMEM/F12 media containing soybean trypsin inhibitor. Resuspended cells are counted by hemocytometer and then pelleted and kept on ice for seeding 3D-rBM cultures. Cells are resuspended with ice cold Matrigel™ to the desired density and introduced into the rMTS device by syringe as described above. The microfluidic device is incubated at 37° C. to solidify the Matrigel™, then liquid media is added to the wells over the 3D cell-ECM layer. The microfluidic devices is incubated in a standard humidified incubator in 5% CO₂ in air. Liquid media is replenished as frequently as necessary to maintain cell viability and allow organoid formation. The MCF-10A organoids are generally fully formed and growth arrest 6-8 days after seeding (12).

Some of the key parameters that are important in adapting conventional cell culture methods for the microfluidic device include for example, the number of cells in seed inoculum and the volume/thickness of ECM layers, and frequency of media changes. The number of cells needed to generate fibroblast monolayers and epithelial organoids in the microfluidic chamber is determined empirically; as a guideline it is useful to scale down from the seeding cell densities used for 24 well plates (11). The minimal thickness (<100 μM) is most desirable for microscopic examination, and well structured epithelial organoids will form even when only partially imbedded in ECM (3, 12). However, the ability to separately address the stromal and epithelial compartments may be compromised if the layers are too thin. Also, the surface area to volume ratio is much higher in a microfluidic device than in conventional cell culture, so media and/or oxygen can be exhausted more quickly, requiring more frequent changes or even a constant flow.

While the present invention has now been described and exemplified with some specificity, those skilled in the art will appreciate the various modifications, including variations, additions, and omissions that may be made in what has been described. Accordingly, it is intended that these modifications also be encompassed by the present invention and that the scope of the present invention be limited solely by the broadest interpretation that lawfully can be accorded the appended claims.

### CITED PUBLICATIONS

1. Leoni A. Kunz-Shughart, Marina Kreutz, and Ruth Knuechel, Int. J. Exp. Path. (1998), Vol. 79: pp 1-23.

2. Derek Radisky, Carmen Hagios, and Mina J. Bissel, Seminars in Cancer Biology, (2001), Vol. 12: pp 97-104.

3. Malathy P. V. Shekhar, Jill Werdell, Steve J. Santer, Robert J. Pauley, and Larry Tait, Cancer Research (2001), Vol. 61: pp 1320-1326.

4. Douglas Hanahan, and Robert A. Weinnberg, Cell, (2000), Vol. 100: pp 57-70.

5. Hood, J. D. and D. A. Cheresh, Nat Rev Cancer, (2002), 2(2): p. 91-100.

6. Yu, J. L. and J. W. Rak, Breast Cancer Res, (2003), 5(2): p. 83-8.

7. Haslarn, S. Z. and T. L. Woodward, Breast Cancer Res, (2003), 5(4): p. 208-15.

8. Hansen, R. K. and M. J. Bissell, Endocr Relat Cancer, (2000), 7(2): p. 95-113.

9. Wemert, N., et al., Anticancer Res, (2001), 21(4A): p. 2259-64.

10. Moinfar, F., et al., Cancer Res, (2000), 60(9): p. 2562-6.

11. Blaschke, R. J., et al., Methods Enzymol, (1994), 245: p. 535-56.

12. Petersen, O. W., et al., Proc Natl Acad Sci USA, (1992), 89(19): p. 9064-8.

13. Kunz-Schughart, L. A., et al., Exp Cell Res, (2001), 266(1): p. 74-86.

14. Kunz-Schughart, L. A., M. Kreutz, and R. Knuechel, Int J Exp Pathol, (1998), 79(1): p. 1-23.

15. Seidl, P., et al., Int J Cancer, (2002), 102(2): p. 129-36.

16. Shekhar, M. P., J. Werdell, and L. Tait, Cancer Res, (2000), 60(2): p. 439-49.

17. Shekhar, M. P., et al., Cancer Res, (2001), 61(4): p. 1320-6.

18. Zhang, H. Z., et al., Endocrinology, (2002), 143(9): p. 3427-34.

19. Simian, M., et al., Development, (2001), 128(16): p. 3117-31.

20. David J. Beebe, Glannys A. Mensing, and Glenn M. Walker. Annu. Rev. Biomed. Eng. (2002), Vol. 4: pp 261-286.

21. Christopher Khoury, Glennys A. Mensing, and David J. Beebe, Lab Chip, (2002), Vol. 2: pp 50-55.

22. David J. Beebe, Jeffrey S. Moore, Quing Yu, Robin H. Liu, Mary L. Kraft, Byung-Ho Jo, and Chelladurai Devadoss, PNAS, (2000), Vol. 97: pp 13488-13493.

23. Rolf Bjerkvig, 1992, Spheroid Culture in Cancer research, CRC Press.

24. David R. Blatchford, Lynda H. Quarrie, Elizabeth Tonner, Corina McCarthy, David J. Flint, Colin J. Wilde, Journal of Cellular Physiology, (1999), Vol. 181: pp 304-311.

25. Ian K. Glasgow, Henry Chris Zeringue, David J. Beebe, Seong-Jun Choi, Joseph T. Lyman, Natalie G. Chan, Mathew B. Wheeler, IEEE Transactions on Biomedical Engineering (2001), Vol. 48: pp 570-578.

26. H. C. Zeringue, D. J. Beebe, and M. B. Wheeler, Biomedical Microdevices (2001), Vol. 3: pp 219-224.

27. Scott F. Gilbert, 1997, Developmental Biology, Sinauer Associates, Inc.

28. Michael G. Olsen, Joseph M. Bauer, David J. Beebe, Applied Physics Letters (2000), Vol. 76: pp 3310-3313.

29. J. D. Trumball, I. K. Glasgow, D. J. Beebe, R. L. Magin, IEEE Transactions in Biomedical Engineering, (2000), Vol 47: pp 3-7.

30. Khoury, C., G. A. Mensing, and D. J. Beebe, Lab on a Chip, (2002), 2(1): p. 50-55.

31. Drumheller, P. D. a. J. A. H., Journal of Biomedical Materials Research, (1995), 29(2): p. 207-15.

32. Bryant, S. J., C. R. Nuttelman, and K. S. Anseth, Journal of Biomaterials Science-Polymer Edition, (2000, 2000), 11(5): p. 439-457.

33. Debnath, J., S. K. Muthuswamy, and J. S. Brugge, Methods, (2003), 30(3): p. 256-68.

34. Muthuswamy, S. K., et al., Nat Cell Biol, (2001), 3(9): p. 785-92.

35. Zantek, N. D., et al., Clin Cancer Res, (2001), 7(11): p. 3640-8.

36. Wang, F., et al., J Natl Cancer Inst, (2002), 94(19): p. 1494-503.

1. A microscale fluid handling system comprising, a microfluidic device and at least one three dimensional multicellular surrogate tissue assembly, wherein the device is used for initiating, culturing, manipulating, and assaying each tissue assembly of the system.

2. The system of claim 1, wherein the device comprises at least one microfluidic channel; and at least one chamber, wherein the chamber contains at least one multicellular surrogate assembly; and wherein fluid medium flows through each channel and chamber of the device.

3. The system of claim 1, wherein at least one multicellular surrogate assembly is a spheroid.

4. The system of claim 1, wherein the device is fabricated using a method selected from the group consisting of liquid phase photopolymerization, elastomeric micromolding, and silicon/glass micromachining.

5. The system of claim 4, wherein the device is the fabricated device comprises chemical compounds selected from the group consisting of polydimethylslane, isoboronyl acrylate, polyethylene glycol diacrylate, hydrogel, glass, and silicon.

6. The system of claim 1, wherein the spheroid is used to model tumorigenic processes through epithelial cell invasion of the stroma, epithelial-mesenchymal transition, or angiogenesis.

7. The system of claim 6, wherein the spheroid is used to model the transition from ductal carcinoma in situ to invasive carcinoma in mammary tumorigenesis.

8. The system of claim 6, wherein the spheroid is used to model a medical condition.

9. The system of claim 6, wherein the condition is cancer.

10. The system of claim 6, wherein the cancer is breast cancer.

11. A microfluidic device for initiating, culturing, manipulating, and assaying multicellular surrogate tissue assemblies comprising at least one microfluidic channel; at least one chamber; and at least one spheroid; and wherein fluid medium flows through each channel and chamber of the device.

12. The device of claim 11, wherein at least one spheroid includes a non-control spheroid and a control spheroid.

13. The device of claim 12, comprising at least one port for the introduction and extraction of the non-control spheroid.

15. The device of claim 14, wherein the fluid medium has a flow rate that is reversible, continuous or pulsed.

16. The device of claim 15, wherein the device comprises at least one obstacle for holding the spheroid in place while maintaining a flow of medium past the spheroid.

17. The device of claim 16, further comprising a spheroid-sorting obstacle for sorting the spheroids, wherein the sorting is conducted by size.

18. The device of claim 15, wherein at least one channel is used for establishing a multi-component laminar flowing stream of medium, wherein at least two components of the medium are capable of contacting different portions of the spheroid.

19. The device of claim 11, wherein the spheroid is used to model tumorigenic processes through epithelial cell invasion of the stroma, epithelial-mesenchymal transition, or angiogenesis.

20. The system of claim 11, wherein the spheroid is used to model the transition from ductal carcinoma in situ to invasive carcinoma in mammary tumorigenesis.

21. The device of claim 11, wherein the spheroid is used to model a medical condition.

22. The device of claim 21, wherein the condition is cancer.

23. The device of claim 22, wherein the cancer is breast cancer.

24. The device of claim 1 1, wherein at least one chamber or at least one channel or a combination thereof are used to initiate the formation and growth of the spheroid.

25. The device of claim 11, wherein at least one chamber or at least one channel or a combination thereof are seeded with fibroblasts.

26. The device of claim 25, wherein the fibroblast seeded chamber and channel can be used to culture a spheroid.

27. The device of claim 11, wherein the spheroid is a heterotypic spheroid.

28. The device of claim 11, wherein the spheroid is comprised of a cell.

29. The device of claim 28, wherein the cell is selected from the group consisting of a fibroblast, an endothelial, a normal epithelial, and a preneoplastic epithelial cell type.

30. A microfluidic device for initiating, culturing, manipulating, and assaying multicellular surrogate tissue assemblies comprising two adjacent chambers wherein each chamber contains cells representing a different tissue compartment, and wherein each chamber contains a fluid medium specific for a tissue compartment.

31. The device of claim 25, wherein the cell is an epithelial cell, a stromal cell, or a coculture of two different cells.

32. The device of claim 31, wherein the cell is of mammary origin.

33. The device of claim 31, wherein the cell is embedded in an extracellular matrix (ECM) selected from the group consisting of collagen, synthetic or natural ECM mixtures, such as Matrigel™, or a combination thereof.

34. The device of claim 31, wherein the cell type is a combination thereof selected from the group consisting of primary cultures or established cell lines, normal or malignant cells, and cells representing various stages of disease progression.

35. The device of claim 31, wherein the cell type is a mammary cell.

36. A method of using the device of claim 11, to model tumorigenic processes wherein the processes include invasion of stromal compartment by epithelial cells, the epithelial-mesenchymal transition, or angiogenesis.

37. The method of claim 36, wherein the tumorigenic processes is the transition from ductal carcinoma in situ to invasive carcinoma in breast cancer.

38. The method of claim 36, wherein the spheroid serves as a model for neoplastic progression.

39. A method of performing high throughput screening of test agents using surrogate tissue assemblies, the method comprising the steps of:
making a microfluidic device including fluid flow channels and chambers;
making surrogate tissue assemblies of multiple cell types of mammalian cells;
placing surrogate tissue assemblies into chambers in the device;
introducing test agents through the fluid flow channels to the surrogate tissue assemblies; and
observing the responses of the surrogate tissue assemblies.

40. The method of claim 39, wherein the responses comprise changes in spheroid proliferation, gene expression, enzyme activity, cell markers, products secreted from the spheroid, an observed change in morphology, tissue invasion and metastasis, or a combination thereof.

41. The method of claim 39, wherein the responses comprise a self sufficiency in growth signals, an insensitivity to growth inhibition, angiogenesis, an evasion of apoptosis, a tissue invasion and metastasis, or a combination thereof.

42. A high throughput screening system for mimicking the reaction of multicellular tissues to test agents, the system comprising;
a microfluidic device having a plurality of fluid flow channels and a plurality of chambers; and
a plurality of surrogate tissue assemblies formed of living mammalian cells, each surrogate tissue assembly located in one of the chambers.
